(19) United States
(12) Patent Application Publication
(10) Pub. No.: US 2007/0042051 A1
(43) Pub. Date: Feb. 22, 2007

(54) Remedy ointment for piles and their manufacturing method

(76) Inventor: Gap-Man Lim, Pusan (KR)

Correspondence Address: JORDAN AND HAMBURG LLP, 122 EAST 42ND STREET, SUITE 4000, NEW YORK, NY, 10168, US

(21) Appl. No.: 10/574,339

(22) PCT Filed: Oct. 19, 2004
(86) PCT No.: PCT/KR04/02680

§ 371 Date: Mar. 30, 2006

(30) Foreign Application Priority Data
Oct. 23, 2003 (KR) 10-2003-0074453

(51) Int. Cl. A61K 35/12

(52) U.S. Cl. 424/520

(57) ABSTRACT

A remedy ointment for piles includes the following steps; forming a base-mixture which include collecting a spawn of a swellfish and a starfish and powdering the same; forming a mixture which is obtained by mixing the powder of a starfish and a swellfish spawn obtained by the above step with a mixture of banana, salt, and water; mixing an alcohol in the vessel contained the mixture formed by the above step; mixing at a heating state which includes heating the vessel filled with the alcohol-mixture obtained by the above step, firing the alcohol in the vessel by indirect transmission of heat and remixing at the fired state; and cooling the mixture to normal temperature by natural cooling. The above remedy ointment for piles has an advantage that it can be easily prepared and is simultaneously convenient to use and easy to store because of preparation in the form of an ointment and has a prominent effect in the treatment of piles.

### TECHNICAL FIELD

The present invention relates to a remedy ointment for piles, more particularly, to a remedy ointment for piles prepared from powder of swellfish spawn as principle ingredient for using as a treatment of internal hemorrhoids, external hemorrhoids, anal fistula and the like.

### BACKGROUND ART

The swellfish is, in general, considered as the most delicious food, but it can sometimes cause damage by toxin due to an inadvertent error in preparation for cookery. The reason is that the swellfish has virulence of brown liquid known as tetrodotoxin. For cooking a swellfish, therefore, a culinarian having a professional skill can only cook a swellfish. Namely, it must be demanded to give attention because a toxin of a swellfish is virulently poisonous.

A tetrodotoxin is not decomposed at high temperature more than 300° C., and do not lose virulence in any seasoning having a severe acidity, and it is known to lose a virulence slowly in hydrochloric acid only. The above poison is more stronger 13 times than potassium cyanide(KCN) and it is known as a virulent poison such that can kill an adult having weight of 50 kg on the spot by only the amount of 0.5 mg.

However, it has been tried to use the above virulent poison of a swellfish for remedy and the toxicity of a swellfish takes charge of an important role in the part of a nervous treatment in modern medical science. In other words, the tetrodotoxin among the toxicity of a swellfish is diluted and then the diluted tetrodotoxin has been used as an anodyne for a neuralgia, an arthritis, and a rheumatism patient, and it is known as to have sedation effect on a shock, an asthma, and a convulsions of tetanus and the like.

Korean Patent Application Publication No. 2002-64807 that is filed by the present applicant with the Korean Patent Office disclosed a remedial agent for piles using swellfish spawn according to the known art.

The above disclosed art comprises; heat treatment and drying step which treat a spawn collected from a swellfish at 0° C.˜30° C. during 50˜150 days; powder forming step which powder by grinding the above heat treated swellfish spawn; and mixing step forming a mixture which blend the above powder of a swellfish spawn with sodium chloride, and it further comprise a fumigating step which fumigate the above mixture using an alcohol. However, the above disclosed art has a drawback which is not only complicate for preparing process, but also the process is troublesome to use because it demand a fumigating step to treat piles.

### DISCLOSURE OF INVENTION

### Technical Problem

It is an object of the present invention to provide a manufacturing method for a remedy ointment for piles which can be easily prepared and is simultaneously convenient to use and easy to store because of preparation in the form of ointment-type. It is also an object of the present invention to provide a remedy ointment for piles produced by the above process.

### TECHNICAL SOLUTION

In order to achieve the above object, the present invention provides a remedy ointment for piles and a manufacturing method for the same, in which a manufacturing method for a remedy ointment for piles is technical gist of comprising following steps; forming a base-mixture which consist of collecting an spawn of a swellfish and a starfish and powdering the same; forming a mixture which is obtained by mixing the powder of a starfish and a swellfish spawn obtained by the above step with a mixture of banana, salt, and water; mixing an alcohol in the a vessel contained the mixture formed by the above step; mixing at heating state which consist of heating the vessel filled with the alcohol-mixture obtained by the above step, firing the alcohol in the vessel by indirect transmission of heat and remixing at the fired state; and, cooling the mixture at the normal temperature by natural cooling.

The present invention provides also as technical gist a remedy ointment for piles which is prepared by step of forming a powder from an spawn of a swellfish and a starfish, forming a mixture by mixing the above powder with a mixture of banana, salt, and water in a vessel, mixing an alcohol in the above vessel; mixing at heating state which consist of heating the vessel filled with the alcohol-mixture, firing the alcohol in the vessel by indirect transmission of heat and remixing at the fired state, and then cooling the mixture at the normal temperature.

Wherein, the above spawn of a swellfish and a starfish are dried by heat-treatment at 0° C.˜30° C. during 50˜150 days and grinded to form powder, and the mixing ratio by weight of swellfish spawn: starfish: banana: salt: water for the above mixture is preferably in the range of 1:0.1˜0.5:10˜20:0.05˜0.1:2˜5, it is more preferable that a powder of a charonia sauliae is further included to the above mixture.

According to the above described present invention, the remedy ointment for piles has an advantage that it can be easily prepared and is simultaneously convenient to use and easy to store because of preparation in the form of ointment-type, and has prominent effect in treatment of a piles.

The preferred embodiments of the present invention is best understood with reference to the accompanying drawings, wherein:

FIG. 1 is a flowchart presenting a preparing process according to the present invention.

As shown at a figure, the preparing process of a remedy ointment for piles according to the present invention consists of a step forming a base-mixture; a step forming a mixture; a step mixing an alcohol; a step mixing at heating state; and, a step cooling the mixture.

First, it is explained in detail on the step forming a base-mixture.

The above step forming a base-mixture is a step of preparing a powder of a swellfish spawn and a starfish.

A powder of a swellfish spawn is prepared by using a swellfish spawn containing a tetrotoxin.

Before everything, a swellfish spawn is collected from a swellfish. At this time, the swellfish that can be used is preferably Lagocephlus lunaris spadiceus, Takifugu xanthopterus, Tetraodonitidae, Lagocephlus lunaris and the like, in the aspect of efficacy, Tetraodonitidae is most preferable and a swellfish spawn is collected in the aggregated state each other.

The collected swellfish spawn is subjected a drying process by heat treatment, the above drying process by heat treatment continued at a temperature of 0° C.˜30° C. during from 50 days to 150 days.

It is not also harmful even though the above drying process by heat treatment is processed in a shadow at normal temperature.

Provided a swellfish spawn in the aggregated state each other is subjected the above drying process by heat treatment, the moisture contained in a swellfish spawn is entirely removed.

Wherein the present invention, the reason of executing the above drying process at low or normal temperature is to prevent a breakup of a mass of a swellfish spawn and to prevent a lose of a toxicity contained in a swellfish spawn.

In the process of powdering a swellfish spawn subjected the above drying process, it can be grinded to powder by giving a prescribed impact using a hammer made of rubber. Wherein, the above swellfish spawn can be allowed to disaggregate a unit of granule.

The above powder of a starfish is also prepared the starfish containing a tetrotoxin. A starfish is allowed to dry at a temperature of 0° C.˜30° C. during from about 50 days to about 150 days. And then the dried starfish is allowed to be grinded to powder by giving a prescribed impact to it using a hammer.

Even though we explained in the above a process drying a swellfish spawn and a starfish and then powdering the same, it is not also harmful to powder by the milling method using mixer etc. at the state of not drying a swellfish spawn and a starfish and this method is also included the scope of the present invention.

With completion of the above procedure, the preparing powder of a swellfish spawn and a starfish is accomplished.

Next, a step forming mixture is followed. At this step, the powder of a swellfish spawn and a starfish prepared by the above step forming a base-mixture is introduced into a vessel, and a certain amount of banana, salt, and water is also introduced to the above vessel, and then the content in the vessel is mixed each other.

If necessary, a powder of a charonia sauliae can be further included to the above mixture with a desired amount. At this time, the mixing ratio by weight of swellfish spawn: starfish: banana: salt: water: charonia sauliae for the above mixture is in the range of 1:0.1˜0.5:10˜20:0.05˜0.1:2˜5:0.1˜0.5.

With completion of the above procedure forming mixture, the step mixing alcohol is followed. At this step, a certain amount of alcohol is added to the vessel received the above mixture, and then the content in the vessel is mixed each other.

Next, a step mixing at heating state is followed. This step is proceeded the way that the mixture received in the vessel, mixed with alcohol, is heated with indirect heat transmission way.

Provided that the above mixture is heated, alcohol is fired, and then at fired state the mixture mixed within the vessel is remixed with agitation. The ignited fire is maintained a fired state till alcohol is entirely consumed. With consumption of alcohol, the fire is out.

With completion of a step mixing at heating state by the above procedure, the cooling step is followed. This cooling step proceeded the way that cool at normal temperature by natural cooling.

With completion of the above cooling step, the remedy ointment for piles according to the present invention is obtained in the form of ointment having hard gel state.

The remedy ointment for piles obtained by the above procedure can be used by the way that put it on piles and anal fistula and the like.

### ADVANTAGEOUS EFFECTS

The remedy ointment for piles according to the present invention obtained by the above procedure can be easily prepared and is simultaneously convenient to use and easy to store because of preparation in the form of ointment-type.

### DESCRIPTION OF DRAWINGS

Other objects and aspects of the present invention will become apparent from the following description of embodiments with reference to the accompanying drawing in which:

FIG. 1 is a flowchart presenting a preparing process of the remedy ointment for piles according to the present invention.

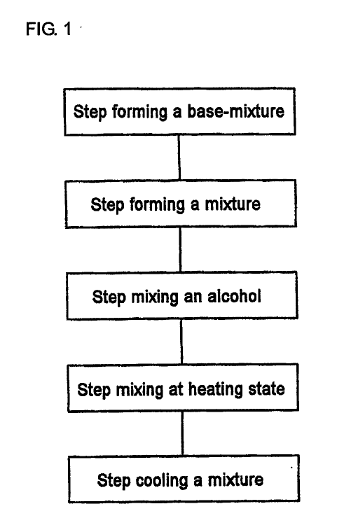

### BEST MODE

The manufacturing method for a remedy ointment for piles according to the present invention is preferably constructed comprising following steps; forming a base-mixture which consist of collecting an spawn of a swellfish and a starfish and powdering the same; forming a mixture which is obtained by mixing the powder of a starfish and a swellfish spawn obtained by the above step with a mixture of banana, salt, and water; mixing an alcohol in the a vessel contained the mixture formed by the above step; mixing at heating state which consist of heating the vessel filled with the alcohol-mixture obtained by the above step, firing the alcohol in the vessel by indirect transmission of heat and remixing at the fired state; and, cooling the mixture at the normal temperature by natural cooling.

The above step forming a base-mixture can be executed in the way that spawn of a swellfish and a starfish are dried by heat-treatment at 0° C.˜30° C. during 50˜150 days and grinded to form powder, and the mixing ratio by weight of swellfish spawn: starfish: banana: salt: water for the above mixture is preferably in the range of 1:0.1˜0.5:10˜20:0.05˜0.1:2˜5, a powder of a charonia sauliae is also further included at the step forming mixture.

The remedy ointment for piles prepared by the above manufacturing method can be formed such that a spawn of a swellfish and a starfish are dried and pulverized, then it is introduced into a vessel and banana, salt, and water are added to the vessel to form a mixture, and alcohol is mixed in the above vessel, and the mixture is remixed at the fired state by ignition of alcohol by indirect transmission of heat, and then the mixture is cooled at the normal temperature, it is possible to further include a charonia sauliae to the above mixture.

1. A method for manufacturing a remedy ointment for piles comprising the steps of; forming a base-mixture which includes collecting a spawn of a swellfish and a starfish and powdering the same; forming a mixture which is obtained by mixing the powder of the starfish and the swellfish spawn obtained by the above step with a mixture of banana, salt, and water; mixing an alcohol in a vessel containing the mixture formed by the above step; mixing at heating state which comprises heating the vessel filled with the alcohol-mixture obtained by the above step, firing the alcohol in the vessel by indirect transmission of heat and remixing at the fired state; and, cooling the mixture to a normal temperature by natural cooling.

2. The method according to claim 1, wherein said step forming a base-mixture is executed in the way that the spawn of a swellfish and a starfish are dried by heat-treatment at 0° C.˜30° C. during 50˜150 days.

3. The method according to claim 1 or 2, wherein the mixing ratio by weight of swellfish spawn: starfish: banana: salt: water for the said mixture is in the range of 1:0.1˜0.5:10˜20:0.05˜0.1:2˜5.

4. The method according to claim 3, wherein, at said step forming a mixture, powder of a charonia sauliae is further included.

5. The remedy ointment for piles that is prepared by drying and pulverizing a spawn of a swellfish and a starfish, then introducing them into a vessel, adding banana, salt, and water to the vessel to form a mixture, mixing alcohol in the above vessel, remixing the mixture at a fired state by ignition of alcohol by indirect transmission of heat, and then cooling the mixture to normal temperature.

6. The remedy ointment for piles according to claim 5, further including a charonia sauliae added to the above mixture.
